(19) United States
(12) Patent Application Publication
(10) Pub. No.: US 2009/0163896 A1
(43) Pub. Date: Jun. 25, 2009

(54) GRASPING TOOL

(75) Inventors: Munetaka Kumate, Kurume-shi (JP); Shuro Hayashi, Hiroshima-shi (JP)

Correspondence Address: ROBERTS MLOTKOWSKI SAFRAN & COLE, P.C.;Intellectual Property Department, P.O. Box 10064, MCLEAN, VA, 22102-8064, US

(73) Assignee: JMS CO., LTD., Hiroshima-shi, Hiroshima (JP)

(21) Appl. No.: 12/090,351

(22) PCT Filed: Oct. 17, 2006
(86) PCT No.: PCT/JP2006/320616

§ 371 Date: Apr. 15, 2008

(30) Foreign Application Priority Data
Oct. 17, 2005 (JP) 2005-301444

(51) Int. Cl. A61B 17/00

(52) U.S. Cl. 606/1; 604/93.01

(57) ABSTRACT

A wire is folded at the central part in the longitudinal direction thereof to form a holding member including a first linear portion 17, a return portion 18, and a second linear portion 19. The first linear portion 17 is inserted through a first pipe 13 of a guide member 2 while the second linear portion 19 is inserted through a second pipe 14 thereof. A first operation part 20 and a second operation part 23 are provided at the first linear portion 17 and the second linear portion 19, respectively. The first linear portion 17 and the second linear portion 19 of the holding member 4 are protruded from the distal end of the guide tube 10 to form a loop 3. The second linear portion 19 is protruded from the distal end of the guide tube 10 longer than the first linear portion 17 to change the shape of the loop 3 and to enlarge the loop 3.

### TECHNICAL FIELD

Conventionally, as a holder of this kind, there has been known a holder including a long tubular guide member to be inserted in an organism, a flexible loop for holding a target object, such as a tissue, an indweller, or the like in the organism, and an operation wire for operating the loop (see Patent Document 1, for example). The operation wire is capable of being inserted in the guide member. The loop is formed along a plane, and one end of the operation wire is connected to one end of the loop.

For using the above holder, the operation wire is first operated to accommodate the loop into the guide member from one end of the guide member. Under this state, the guide member is inserted from the one end part thereof up to the vicinity of a target object in an organism, and then, the operation wire is operated to protrude out the loop accommodated in the guide member from the one end of the guide member. When the loop is moved within the organism by operating the operation wire to position the target object within the loop and is pulled into the guide member, a part on the operation wire side of the loop is gradually accommodated into the guide member to reduced the diameter of the loop, thereby allowing the loop to hold the target object.

Patent Document 1: Japanese Unexamined Patent Application Publication 2000-271146

### DISCLOSURE OF THE INVENTION

### Problems that the Invention is to Solve

The inside of the organism is so complicated that it is difficult in some cases to position the guide member at the vicinity of the target object for positioning, as in the Patent Document 1, the loop at the vicinity of the target object by inserting the guide member into the organism. When the guide member is difficult to be positioned at the vicinity of the target object, it is necessary to operate the operation wire for positioning the loop at the vicinity of the target object.

In the holder in Patent Document 1, however, since the loop is merely formed along a plane and connected to one end of the operation wire, the shape of the loop when completely protruded out from the guide member is substantially fixed. For this reason, the shape and the size of the loop cannot be changed to those allowing the loop to hold the target object easily. Further, in the case where the holder holds a target object within a blood vessel, for example, the loop may move or change in shape by the bloodstream in the blood vessel even with the operation wire not operate to make it difficult to hold the target object.

In short, it is difficult for the holder in Patent Document 1 to hold a target object when it is difficult to insert the guide member up to the vicinity of the target object or when the target object is to be held within a blood vessel, so that it takes much time for treatment, thereby invading a patient severely.

The present invention has been made in view of the foregoing and has its object of reducing invasion on a patient by changing the shape and the size of a loop within an organism for allowing the loop to hold a target object readily.

### Means for Solving the Problems

In order to achieve the above object, in the present invention, a loop is formed of a holding member including a first linear portion, a second linear portion, and a return portion continuing from each one end of the linear portions and the first linear portion and the second linear portion are made operable from the outside of an organism.

Specifically, in a first aspect of the invention, a holder includes: a long tubular guide member to be inserted at one end part thereof into an organism and to be protruded at the other end part thereof from the organism; and a long holding member of which one end part is inserted in the guide member to be guided into the organism and which forms a loop for holding a target object within the organism, wherein the holding member includes: a first linear portion extending from the one end part of the holding member and protruded from the other end part of the guide member; a return portion continuing from one end part of the first linear portion and returned toward the other end part of the guide member; and a second linear portion continuing from the return portion and extending toward and protruded from the other end part of the guide member, and a first operation part and a second operation part which are to be held and operated by an operator are respectively provided at parts of the first linear portion and the second linear portion which are protruded from the other end part of the guide member.

In the above arrangement, when the first operation part and the second operation part are operated to protrude the one end part of the holding member from the one end part of the guide member with the one end part of the guide member inserted within the organism, the loop is formed by the return portion and each one end part of the linear portions of the holding member. In the case where the it is difficult to insert the one end part of the guide member up to the vicinity of the target object within the organism, the first operation part and the second operation part are moved to protrude the linear portions long from the one end part of the guide member, thereby allowing the loop to approach the target object and being large. When only the second operation part is moved without moving the first operation part with the linear portions protruded from the one end part of the guide member to protrude the second linear portion long from the one end part of the guide member, the second linear portion curves away from the first linear portion to change its shape and be large because the linear portions are connected to each other via the return portion. In reverse, when only the first operation part is moved to protrude the first linear portion long from the one end part of the guide member, the first linear portion changes its shape and becomes large. Further, when the second operation is twisted without moving the first operation part, the twisting force is transmitted from the one end part of the second linear portion to the return portion to twist the loop entirely, thereby allowing the loop to be in a three-dimensional shape. Similarly, when only the first operation part is twisted, the loop is in a three-dimensional shape. Twisted operation of the first operation part and the second operation part changes the direction of the loop.

In short, operation of the first operation part and the second operation part outside the organism with the guide member and the one end part of the holding member inserted within the organism can change the position, the shape, the size, and the direction of the loop.

Referring to a second aspect of the present invention, in the first aspect, the other end part of the guide member branches into a first pipe through which the other end part of the first linear portion is inserted and a second pipe through which the other end part of the second linear portion is inserted.

In the above arrangement, the other end part of the first linear portion and the other end part of the second linear portion are protruded from the pipes different from each other. This enables separation of the first operation part and the second operation part from each other.

Referring to a third aspect of the present invention, in the second aspect, the first pipe is provided with first fixing means for switching the first linear portion to and from a state that the other end part thereof is fixed to the first pipe from and to a state that the fixed state is released.

With the above arrangement, in the case where it is desired to change the shape, the size, or the like of the loop slightly by operating only the second operation part, the first linear portion can be fixed to the first pipe by the first fixing means to be immovable.

Referring to a fourth aspect of the present invention, in the third aspect, the second pipe is provided with second fixing means for switching the second linear part to and from a state that the other end part thereof is fixed to the second pipe from and to a state that the fixed state is released.

With the above arrangement, in the case where it is desired to change the shape, the size, or the like of the loop slightly by operating only the first operation part, the second linear portion can be set to be immovable.

Referring to a fifth aspect of the present invention, in the first aspect, the first operation part and the second operation part are provided with identification parts for identifying the operation parts.

With the above arrangement, the operator can identify the first operation part and the second operation part effortlessly and correctly, thereby enabling secured operation of a desired operation part.

Referring to a sixth aspect of the present invention, in the first aspect, the guide member is divided into an insertion part to be inserted into the organism and a protruded part to be protruded outside the organism, and the insertion part is softer than the protruded part.

With the above arrangement, the inserted part of the guide member in inserting the guide member into the organism deforms readily along the shape of the inside of the organism.

In a seventh aspect of the present invention, an aspirator for aspirating a liquid in the guide member is connected to the guide member.

Referring to an eighth aspect of the present invention, in the first aspect, an insufflator for infusing a chemical solution into the guide member is connected to the guide member.

Referring to a ninth aspect of the present invention, in the first aspect, the guide member is bent at a part on the one end part side thereof.

Referring to a tenth aspect of the present invention, in the first aspect, a mark made of an X-ray non-transmissive material is provided at the one end part of the holding member.

Referring to an eleventh aspect of the present invention, in the first aspect, the inside of the guide member is divided into a room through which the first linear portion of the holding member is inserted and a room through which the second linear portion thereof is inserted.

### Effect of the Invention

In the first aspect of the present invention, the holding member forming the loop for holding the target object within the organism includes the first linear portion, the second linear portion, and the return portion continuing from the linear portions and the operation parts for the operator are provided at the first linear portion and the second linear portion, so that the loop can be changed in position, shape, size, and direction easily from the outside of the organism. This attains easy holding of the target object even in the case where it is difficult to insert the guide member up to the vicinity of the target object within the organism or the case where the target object within a blood vessel is to be held, thereby reducing invasion on the patient.

In the second aspect of the present invention, the other end part of the guide member branches into the first pipe through which the first linear portion is inserted and the second pipe through which the second linear portion is inserted to thus separate the first operation part and the second operation part from each other. This facilitates independent operation of the two operation parts, thereby achieving favorable operability.

According to the third aspect of the present invention, the first fixing means fixes the first linear portion to the first pipe to facilitate slight change in shape, size, and the like of the loop by operation of only the second operation part.

According to the fourth aspect of the present invention, the second fixing means fixes the second linear portion to the second pipe to facilitate slight change in shape, size, and the like of the loop by operating only the first operation part.

In the fifth aspect of the present invention, the identification parts are provided at first operation part and the second operation part to achieve further favorable operability.

In the sixth aspect of the present invention, the guide member is divided into the insertion part to be inserted into the organism and the protrusion part to be protruded outside the organism and the insertion part is set softer than the protrusion part, thereby further reducing invasion on the organism.

In the seventh aspect of the present invention, the guide member is connected to the aspirator, thereby enabling aspiration of blood and the like in the guide member.

In the eighth aspect of the present invention, the insufflator for a chemical solution is connected to the guide member, so that an anesthetic, for example, can be infused into the organism through the guide member.

According to the ninth aspect of the present invention, change in direction of the one end part of the guide member readily changes the direction of the one end part of the holding member.

According to the tenth aspect of the present invention, under radiography with the one end part of the holding member inserted in the organism, the operator can recognize the position of the one end part of the holding member to enable safe operation of the holding member.

In the eleventh aspect of the present invention, the inside of the guide member is divided into the room through which the first linear member is inserted and the room through which the second linear portion is inserted, so that one of the linear portions is prevented from obstructing the other linear portion in moving the other linear portion, thereby facilitating the operation.

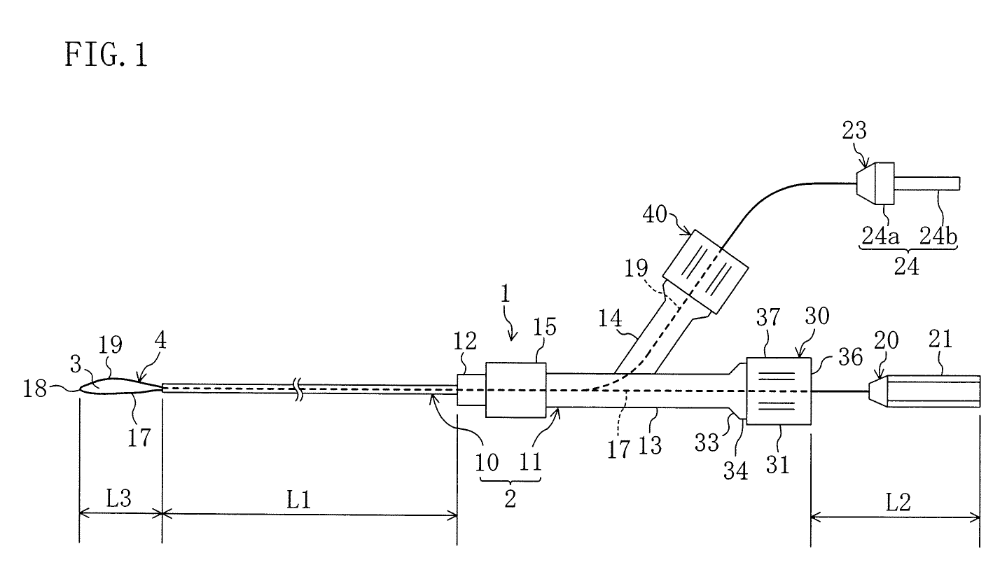

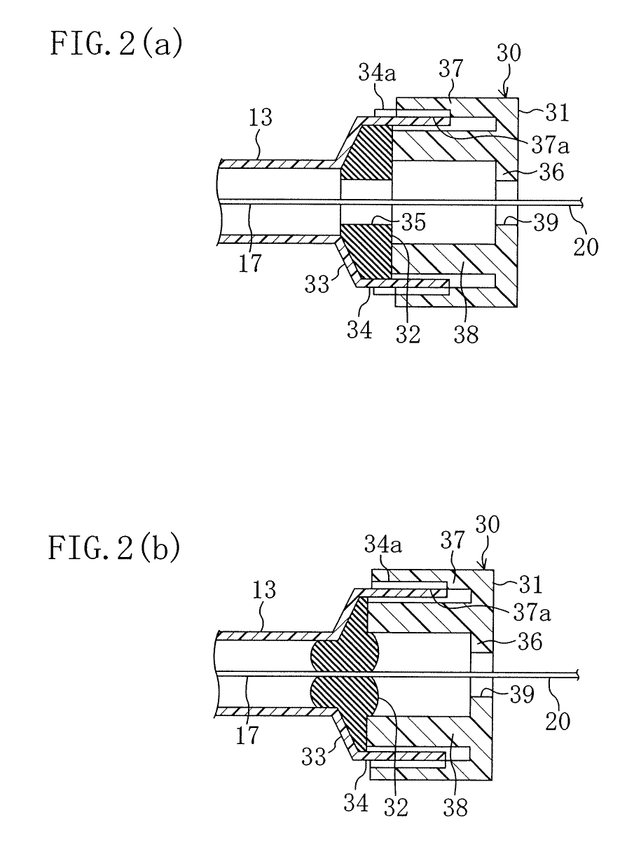

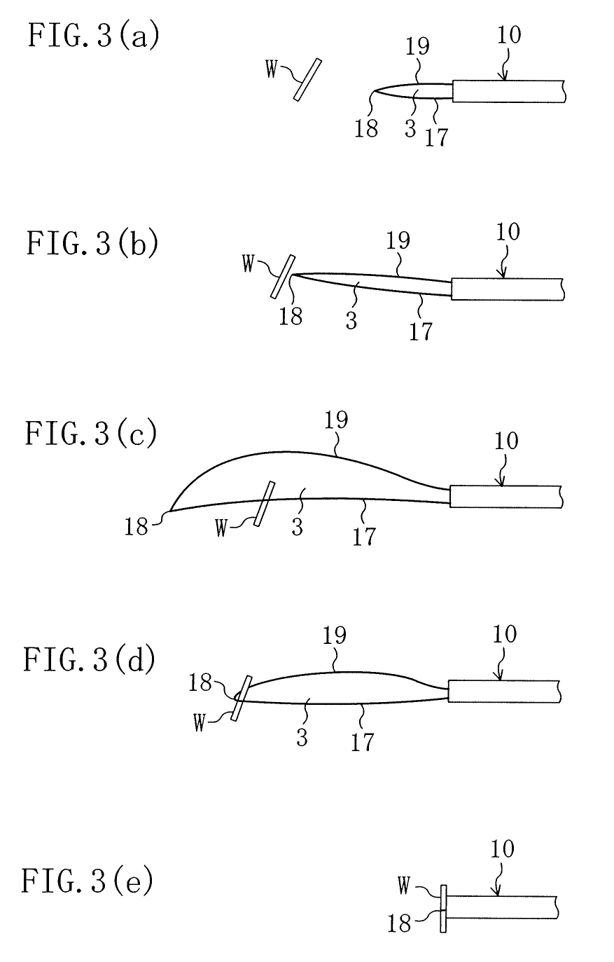

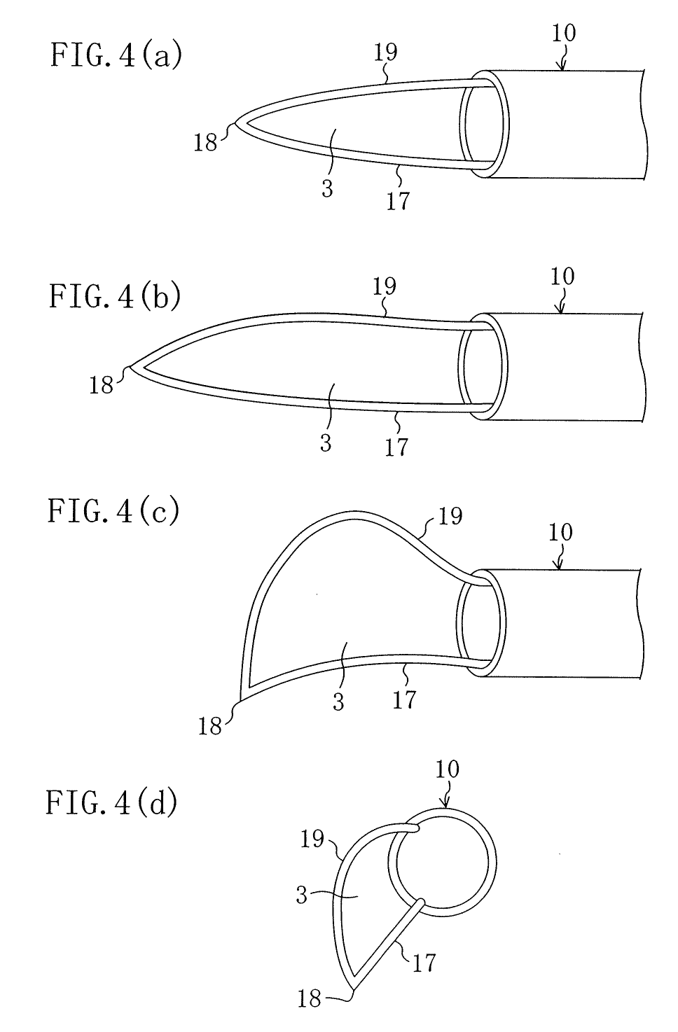

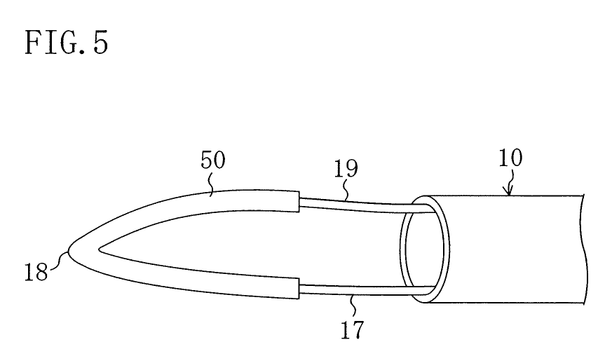

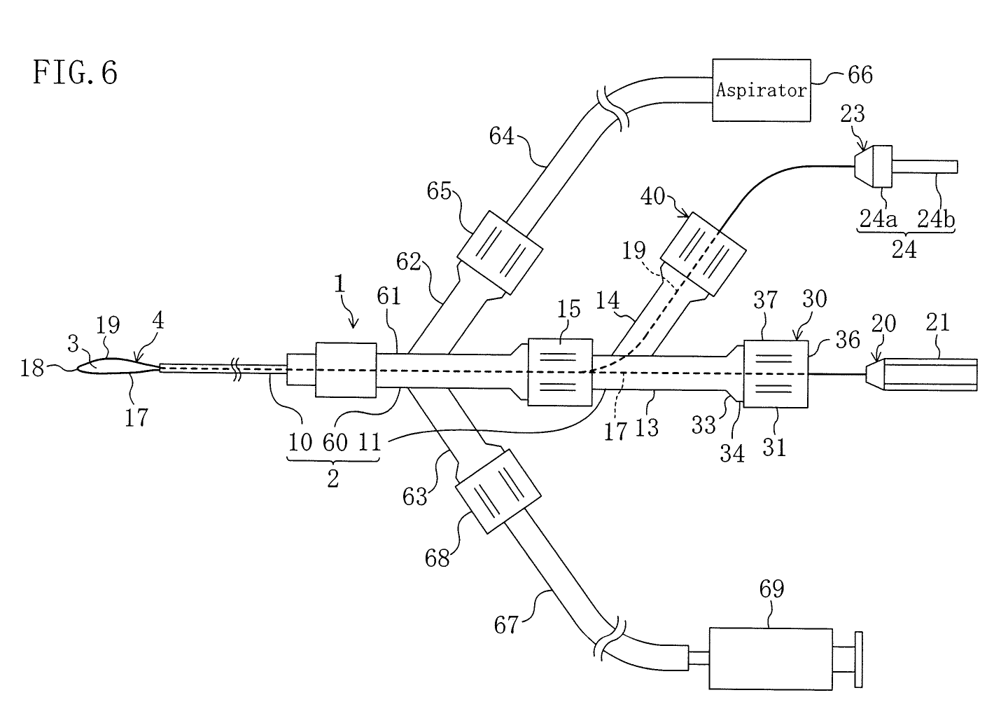

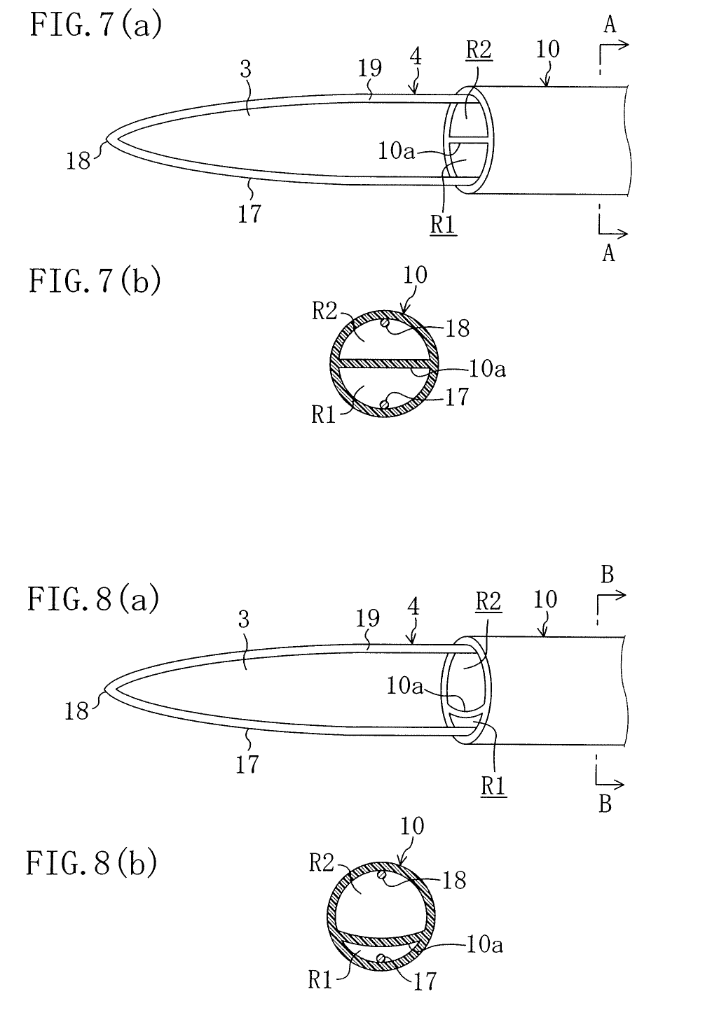

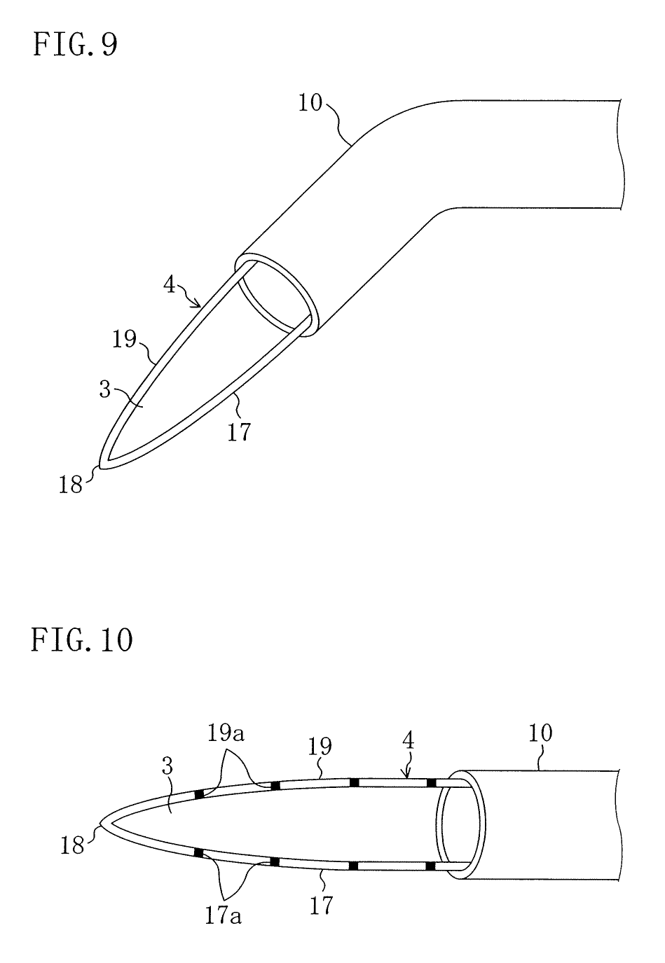

### BRIEF DESCRIPTION OF THE DRAWINGS

[FIG. 1] FIG. 1 is a side view of a holder in accordance with an embodiment of the present invention.

[FIG. 2] FIG. 2 is a sectional view showing a construction of a first fixing mechanism.

[FIG. 3] FIG. 3 is a diagram for explaining a scheme for holding a stent within a blood vessel.

[FIG. 4] FIG. 4(a) is a view showing a state where a holding member is protruded short from a guide tube; FIG. 4(b) is a view showing a state where the holding member is protruded long form the guide tube; FIG. 4(c) is a view showing a state where a second linear portion is protruded longer than a first linear portion; and FIG. 4(d) is a view showing a state where the second linear portion is twisted when viewed from the distal end of the guide tube.

[FIG. 5] FIG. 5 is a view corresponding to FIG. 4(b) in accordance with Modified Example 1 of the embodiment.

[FIG. 6] FIG. 6 is a view corresponding to FIG. 1 in accordance with Modified Example 2 of the embodiment.

[FIG. 7] FIG. 7 relates to Modified Example 3 of the embodiment, wherein FIG. 7(a) is a view corresponding to FIG. 4(b) and FIG. 7(b) is a sectional view taken along the line A-A in FIG. 7(a).

[FIG. 8] FIG. 8 relates to Modified Example 4 of the embodiment, wherein FIG. 8(a) is a view corresponding to FIG. 4(b) and FIG. 8(b) is a sectional view taken along the line B-B in FIG. 8(a).

[FIG. 9] FIG. 9 is a view corresponding to FIG. 4(b) in accordance with Modified Example 5 of the embodiment.

[FIG. 10] FIG. 10 is a view corresponding to FIG. 4(b) in accordance with Modified Example 6 of the embodiment.

### EXPLANATION OF REFERENCE NUMERALS

1 holder

2 guide member

4 holding member

13 first pipe

14 second pipe

17 first linear portion

18 return portion

19 second linear portion

20 first operation part

21 first cylindrical part (identification part)

23 second operation part

24 second cylindrical part (identification part)

30 first fixing mechanism (first fixing means)

41 second fixing mechanism (second fixing means)

W target object

### BEST MODE FOR CARRYING OUT THE INVENTION

An embodiment of the present invention will be described below in detail with reference to the accompanying drawings. The following description of the preferred embodiment is a mere substantial example and is not intended to limit the present invention, applicable objects, and usage thereof.

FIG. 1 shows a holder 1 in accordance with one embodiment of the present invention. The holder 1 is used for holding a target object within the body of a patient and taking it out from the body. The holder 1 includes a long tubular guide member 2 formed so as to be inserted at one end part thereof into the body and be protruded at the other end part thereof outside the body and a long holding member 4 forming a loop 3 for holding a target object within the body. The holding member 4 is inserted in the guide member 2 to be guided into the patient's body.

The guide member 2 includes a long and small-diameter guide tube 10 on the side to be inserted into the body and a branch pipe 11 on the side to be protruded from the body. The guide tube 10 is made of a soft resin material so as to be readily curved and deformed at insertion into the body. The length, the outer diameter, and the inner diameter of the guide tube 10 may be changed according to a part where the holder 1 is used, a kind of the target object, the build, the age and the sex of the patient, and the like. At the end on the branch pipe 11 side of the guide tube 10, a tubular joint 12 is provided to be connected to the branch pipe 11. A radially protruding and peripherally extending stripe protrusion (not shown) is formed at the outer periphery of the joint 12.

The branch pipe 11 is in a Y shape of a first pipe 13 extending along the center line of the guide tube 10 and a second pipe 14 branching from the middle part in the longitudinal direction of the first pipe 13. The first pipe 13 and the second pipe 14 are made of a resin material harder than the resin material of the guide tube 10 and is formed integrally with each other so as not to be readily deformed in operation. Openings are formed in the first pipe 13 and the second pipe 14 at the respective ends opposite to the guide tube 10. The branch pipe 11 may be formed of a known Y-shaped connector.

At the end on the guide tube 10 side of the first pipe 13, an expansion part 15 of which outer diameter expands is provided. An engaging groove (not shown) for receiving the stripe protrusion of the guide tube 10 is formed in the inner face of the expansion part 15. When the stripe protrusion is fitted into the engaging groove, the guide tube 10 and the branch pipe 11 are integrated with each other. Accordingly, the guide tube 10 is exchangeable with another guide tube 10 different in length or the like.

The holding member 4 is a long medical wire with a line diameter in the range between 0.3 mm and 0.7 mm, both inclusive, which is folded around the central part in the longitudinal direction thereof. Specifically, the holding member 4 includes: a first linear portion 17 extending from the distal end of the guide tube 10 toward the branch pipe 11; a return portion 18 continuing from the guide tube 10 side end of the first linear portion 17 and folded toward the branch pipe 11; and a second linear portion 19 continuing from the return portion 18 and extending toward the branch pipe 11. The first linear portion 17 and the second linear portion 19 are longer than the length along the center line of the guide member 2. Accordingly, the branch pipe 11 side ends of the first linear portion 17 and the second linear portion 19 protrude outside the openings of the first pipe 13 and the second pipe 14, respectively.

The return portion 18 is formed around the central part in the longitudinal direction of the wire of the holding member 4, namely, is a folded part of the wire. The return portion 18 is so formed to urge the first linear portion 17 and the second linear portion 19 away from each other.

A part of the first linear portion 17 which is protruded from the first pipe 13 serves as a first operation part 20. On the end of the first operation part 20 opposite to the first pipe 13, a first cylindrical part 21 in a substantially cylindrical shape made of a resin material is detachably mounted. A central hole (not shown) is formed in the first cylindrical part 21 so that the first linear portion 17 is inserted therein and is fixed to the first cylindrical part 21. Whereby, the first linear portion 17 is rotated integrally with the first cylindrical part 21 with it prevented from being pulled out from the central hole.

A portion of the second linear portion 17 which is protruded from the second pipe 14 serves as a second operation part 23. On the end of the second operation part 23 opposite to the second pipe 14, a second cylindrical part 24 in a substantially cylindrical shape made of a resin material is detachably mounted. The second cylindrical part 24 includes a large diameter part 24a and a small diameter part 24b so as to be different in shape from the first cylindrical part 21. A central hole (not shown) is formed in the second cylindrical part 24 so that the second linear portion 19 is inserted therein and is fixed to the second cylindrical part 24. Accordingly, the second linear portion 19 is rotated integrally with the second cylindrical part 24 with it prevented from being pulled out from the central hole. Further, the first cylindrical part 21 is different in color of the resin material from the second cylindrical part 24. In other words, the first cylindrical part 21 and the second cylindrical part 24 form identification parts for identifying the first operation part 20 and the second operation part 23.

As the holding member 4, a plurality of holding members different in line diameter, strength, or the like from each other may be prepared. When prepared, one of the holding members 4 which is suitable for a patient can be selected and exchanged. For exchange, the first cylindrical part 21 and the second cylindrical part 24 are taken off from the first linear portion 17 and the second linear portion 19, respectively, and the holding member 4 is pulled out from the distal end of the guide tube 10. Then, another holding member 4 for exchange is inserted from the distal end of the guide tube 10, and the first cylindrical part 21 and the second cylindrical part 24 are mounted to the first linear portion 17 and the second linear portion 19, respectively.

At the end of the first pipe 13 on the side opposite to the guide tube 10, a first fixing mechanism 30 for fixing the first linear portion 17 to the first pipe 13 is provided. The first fixing mechanism 30 is composed of, as shown in FIG. 2, a part of the first pipe 13 on the side opposite to the guide tube 10, a cap member 31 covering the opening of the first pipe 13, and an elastic member 32 inserted in the inside of the first pipe 13 on the side opposite to the guide tube 10. At the part of the first pipe 13 on the side opposite to the guide tube 10, there are provided a tapered face 33 formed so as to enlarge in diameter as it goes toward the end thereof and an annular part 34 continuing from the tapered face 33. A male screw part 34a is formed around the outer periphery of the annular part 34.

The elastic member 32 is made of a readily deformed soft material, for example, silicone rubber or the like. The outer face of the elastic member 32 tightly adheres to the tapered face 33 and the inner face of the annular part 34. In the elastic member 32, a circular hole 35 with a diameter larger than the line diameter of the first linear portion 17 is formed coaxially with the center line of the first pipe 13, and the first linear portion 17 is inserted through the circular hole 35. The cap member 31 is made of a resin material similar to the material of the first pipe 13 and includes a disc part 36 covering the end part of the first pipe 13 and an outer peripheral wall 37 and an inner peripheral wall 38 which are protruded from the first pipe 13 side of the disc part 36. An insertion hole 39 with a diameter larger than the line diameter of the first linear portion 17 is formed in the disc part 36 coaxially with the center line of the first pipe 13. The outer peripheral wall 37 is formed along the outer face of the annular part 34. A female screw part 37a to be in screw engagement with the male screw part 34a of the annular part 34 is formed in the inner face of the outer peripheral wall 37. The inner peripheral wall 38 is separated inward from the inner face of the annular part 34, and the distal end in the protruding direction of the inner peripheral wall 38 is in contact with the elastic member 32.

When the female screw part 37a of the cap member 31 is screwed into the male screw part 34a of the first pipe 13, the end part of the inner peripheral wall 38 of the cap member 38 pushes the elastic member 32 toward the tapered face 33, so that the inner peripheral wall 38 approaches the tapered face 33 of the first pipe 13. The elastic member 32 pushed by the inner peripheral wall 38 is elastically deformed to reduce the inner diameter of the circular hole 35, so that the inner face of the circular hole 35 adheres to the first linear portion 17 tightly. At this time point, the elastic member 32 is pushed against and adheres tightly to the tapered face 33. Thus, the first linear portion 17 is in a state fixed to the first pipe 13.

In reverse, when the cap member 31 fixing the first linear portion 17 is screwed in the direction for detaching the cap member 17 from the first pipe 13, the inner peripheral wall 38 separates from the tapered face 33 so that the elastic member 32 recovers to the original shape. This releases the fixed state of the first linear portion 17.

A second fixing mechanism 40 similarly to the first fixing mechanism 30 is provided at the end of the second pipe 14 on the side opposite to the guide tube 10. The second fixing mechanism 40 fixes the second linear portion 19 to the second pipe 14. The first fixing mechanism 30 and the second fixing mechanism 40 serve first fixing means and second fixing means, respectively, in the present invention.

As the holder 1, child and adult holders each part of which is different in size from each other are prepared. Referring first to the size of each part of the child holder 1, the thickness of the guide tube 10 is set at approximately 3 to 4 Fr (french) so that the guide tube 10 can be easily inserted into a child's body. The length L1 (indicated in FIG. 1) of the guide tube 10 is set at approximately 600 mm. The distance L2 from the first fixing mechanism 30 to the base end of the first cylindrical part 21 is set at approximately 200 mm, and the distance from the second fixing mechanism 40 to the base end of the second cylindrical part 24 is set substantially the same. The length L3 of the loop 3 protruding from the distal end of the guide tube 10 is set at approximately 100 mm. When the protruding length of the loop 3 is set at 100 mm or smaller, insertion into, for example, a heart can be carried out effortlessly.

On the other hand, the size of each part of the adult holder 1 is referred to. The thickness of the guide tube 10 is set at approximately 5 to 6 Fr (french), and the length thereof is set at approximately 1000 mm. The distance from the first fixing mechanism 30 to the base end of the first cylindrical part 21, the distance from the second fixing mechanism 40 to the base end of the second cylindrical part 24, and the length of the loop 3 protruding from the distal end of the guide tube 10 are set substantially the same as those set in the child holder 1.

When the distance from the first fixing mechanism 30 to the base end of the first cylindrical part 21 and the distance from the second fixing mechanism 40 to the base end of the second cylindrical part 24 are set at approximately 200 mm or greater, the operator can operate the first cylindrical part 21 and the second cylindrical part 24 easily from the position apart from the fixed mechanisms 30, 40.

A scheme for using the thus constructed holder 1 will be discussed next with reference to FIG. 3 and FIG. 4. In the present embodiment, description is given about a scheme for taking out a stent W indwelling in a blood vessel (not shown) from the outside of the patient's body by holding it by the holder 1 in the case where the stent W falls off from the blood vessel. The stent W is the target object. This treatment is carried out under radiography.

First, the first fixing mechanism 30 and the second fixing mechanism 40 release the fixing states. The first operation part 20 and the second operation part 23 are operated to pull to accommodate the end parts on the guide tube 10 side of the first linear portion 17 and the second linear portion 19 and the return portion 18 into the guide tube 10. The first linear portion 17, the return portion 18, and the second linear portion 19 can be accommodated into the guide tube 10 by easy deformation of the flexible holding member 4.

Next, the distal end of the guide tube 10 is inserted into the patient's blood vessel together with the holding member 4 and is positioned at the vicinity of the stent W. Then, when the first linear portion 17, the second linear portion 19, and the return portion 18 are protruded from the distal end of the guide tube 10, the end parts on the guide tube 10 side of the first linear portion 17 and the second linear portion 19 separate from each other by the urging force of the return portion 18 as shown in FIG. 3(a) and FIG. 4(a). Accordingly, the end part on the guide tube 10 side of the first linear portion 17, the return portion 18, and the end part on the guide tube 10 side of the second linear portion 19 form the loop 3 for holding the target object.

The size of the loop 3 becomes larger by elongating each protruding length of the first linear portion 17 and the second linear portion 19 from the distal end of the guide tube 10, as shown in FIG. 4(b), while on the other hand becoming smaller by reducing each protruding length thereof. Elongation of each protruding length of the linear portions 17, 19 from the guide tube 10 separates the distal end of the loop 3 from the distal end of the guide tube 10, as shown in FIG. 3(b), while shortening of each protruding length thereof makes the distal end of the loop 3 to approach the distal end of the guide tube 10.

When only the second operation part 23 is moved without moving the first operation part 20 to protrude the second linear portion 19 long from the distal end of the guide tube 10, the second linear portion 19 curves with a point near the return portion 18 as a starting point to separate from the first linear portion 17, thereby changing the shape of the loop 3, as shown in FIG. 3(c) and FIG. 4(c), because the linear portions 17, 19 continue to each other via the return portion 18. When only the second linear portion 19 is protruded long from the guide tube 10 in this way, the loop is enlarged as well. In reverse, when only the first operation part 20 is moved without moving the second operation part 23 to protrude the first linear portion 17 long from the distal end of the guide tube 10, the loop 3 changes in shape and is enlarged similarly. In addition, when one of the linear portions is pulled into the guide tube 10 in the state that the first linear portion 17 and the second linear portion 19 are protruded by substantially the same length from the guide tube 10, the loop 3 changes in its shape and size.

Moreover, when the second operation part 23 is twisted without moving the first operation part 20, the twisting force thereof is transmitted from the second linear portion 19 to the return portion 18. By the twisting force, the entire loop 3 is twisted and deformed to be in a three-dimensional shape from a plane shape, as shown in FIG. 4(d). Twisting only the first operation part 20 also twists the entire loop 3 similarly to deform the loop 3 three-dimensionally. In addition, twisting the first operation part 20 and the second operation part 23 changes the direction of the loop 3.

The first linear portion 17 may be fixed to the first pipe 13 by the first fixing mechanism 30 when it is unnecessary to move the first operation part 20. As well, the second linear portion 19 may be fixed to the second pipe 14 by the second fixing mechanism 40 when it is unnecessary to move the second operation part 23. By making the first linear portion 17 and the second linear portion 19 to be fixable to the first pipe 13 and the second pipe 14, respectively, the shape, the size, and the like of the loop 3 can be changed easily by operating only one of the linear portions.

As described above, operation of the first operation part 20 and/or the second operation part 23 can change the position, the shape, the size, and the direction of the loop 3. Hence, even in the case where it is difficult to position the distal end part of the guide tube 10 at the vicinity of the stent W according to the shape of a blood vessel or the place where the stent W falls off or in the case where the shape of the loop 3 is liable to change by the bloodstream in a blood vessel, it becomes easy to allow the loop 3 to approach the stent W and to position the stent W within the loop 3. To do so, as shown in FIG. 3(c), the loop 3 is set to be relatively large by protruding the first linear portion 17 and the second linear portion 19 long from the distal end of the guide tube 10, as shown in FIG. 3(c), so that the stent W can be positioned within the loop 3 easily.

The operator operates the first operation part 20 and the second operation part 23 with the stent W positioned within the loop 3 to pull the first linear portion 17 and the second linear portion 19 into the guide tube 10. As the first linear portion 17 and the second linear portion 19 are pulled into the guide tube 10, the size of the lop 3 becomes smaller and the vicinity of the distal end of the loop 3 approaches the stent W, as shown in FIG. 3(d). Then, the loop 3 draws the stent W up to the guide tube 10 and the stent W is held at the return portion 18 of the holding member 4, as shown in FIG. 3(e). The guide tube 10 and the holding member 4 are pulled out from the blood vessel with the stent W held for taking the stent W out from the patient's body.

Thus, in the holder 1 in accordance with the present embodiment, the holding member 4 for holding the stent W falling off within the body is composed of the first linear portion 17, the second linear portion 19, and the return portion 18 continuing from the linear portions 17, 19 and the first operation part 20 and the second operation part 23 are provided at the first linear portion 17 and the second linear portion 19, respectively. Accordingly, even in the case where it is difficult to insert the guide tube 10 up to the vicinity of the stent W and in the case where the loop 3 moves or changes in shape by the bloodstream, the stent W can be held effortlessly, thereby reducing inversion on the patient.

Further, the branch pipe 11 is formed in a Y-shape of the first pipe 13 and the second pipe 14 and the first linear portion 17 is inserted through the first pipe 13 while the second linear portion 19 is inserted through the second pipe 14, so that the first operation part 20 and the second operation part 23 separate from each other. This facilitates individual operation of the two operation parts 20, 23 to thus achieve favorable operability.

The first fixing mechanism 30 fixes the first linear portion 17 to the first pipe 13 to facilitate slight change in position and the like of the loop 3 by operating only the second operation part 23. Similarly, the second fixing mechanism 40 fixes the second linear portion 19 to the second pipe 14 to facilitate slight change in position and the like of the loop 3 by operating only the first operation part 20.

The first cylindrical part 21 and the second cylindrical part 24 are provided at the first operation part 20 and the second operation part 23, respectively, to facilitate identification of the operation parts 20, 23, so that the operator can identify the first operation part 20 and the second operation part 23 effortlessly and correctly. This achieves further favorable operability of the holder 1.

The guide member 2 is divided into the guide tube 10 to be inserted into the patient's body and the branch pipe to be protruded outside the body, and the guide tube 10 is set softer than the branch pipe 11, with a result that invasion on the patient can be reduced in inserting the guide tube 10 into the body.

The guide tube 10 and the branch pipe 11 of the guide member 2 are detachably connected to each other, so that the guide tube 10 can be exchanged with another one different in shape with the same branch pipe 11 used.

While the present embodiment describes the case for taking the stent W out from the body with the use of the holder 1, the holder 1 of the present invention may be used for taking out from the body a coil-shaped blocking member for blocking a blood vessel, a coil-shaped blocking member for blocking a foraminulum formed in a heart, or the like when the blocking member is displaced from a desired place, for example. Further, the holder 1 of the present invention can be used for taking out from a heart an electrode lead wire (target object) of a pacemaker when the electrode lead wire is cut due to aging. In the case where an indwelling catheter, which is used for generally-called IVH (intravenous hyperalimentation) to a vein near the heart carried out when a patient is in poor nourishment, is broken and cut by a cardiac valve or the like and a chip (target object) thereof remains in the vein, the chip can be taken out with the use of the holder 1 of the present invention. The holder 1 of the present invention can be used for taking out from the organism any of an indweller, a tissue, and the like within an organism besides the aforementioned target objects.

As in Modified Example 1 shown in FIG. 5, a non-slip member 50 may be provided at a part of the holding member 4 which forms the distal end part of the loop 3. The non-slip member 50 is formed of a tubular rubber, for example, for preventing the target object from slipping and falling off from the loop 3.

The holding member 4 may be formed of, other than a wire, a thin, long, and flexible member capable of transmitting the pushing force in the longitudinal direction thereof when being pushed in the longitudinal direction thereof.

As in Modified Example 2 shown in FIG. 6, a branch pipe 60 may be provided between the guide tube 10 and the branch pipe 11. The branch pipe 60 forms a part of the guide member 2 and includes a first pipe 61, a second pipe 62, and a third pipe 63. Each one end of the second pipe 62 and the third pipe 63 communicates with the first pipe 61. The first pipe 61 communicates with the guide tube 10, and the holding member 4 is inserted through the first pipe 61. A joint 65 connected to an aspiration pipe 64 is formed at the other end part of the second pipe 62. The aspiration pipe 64 is connected to an aspirator 66. Namely, the guide member 2 is connected to the aspirator 66 through the aspiration pipe 64. The aspirator 66 is a known tool which is capable of aspirating liquid or air and which is generally used in the medical field and may be of motor-driven type, manually-operated pump type, or the like.

Further, a joint 68 to which an infusion pipe 67 is connected is formed at the other end of the third pipe 63. The infusion pipe 67 is connected to an insufflator 39, such as a syringe for infusing a chemical solution. Namely, the guide member 2 is connected to the insufflator 69 through the infusion pipe 67. Alternatively, only one of the aspiration pipe 64 and the infusion pipe 67 may be connected to the branch pipe 60. The insufflator 69 may be a motor-driven syringe pump.

According to the holder 1 of Modified Example 2, when the aspirator 66 is operated with the guide tube 10 inserted within an organism, the air in the guide tube 10 can be aspirated. Further, the insufflator 69 can infuse, for example, heparin as an anticoagulant into the guide tube 10 through the infusion pipe 67, the third pipe 63, and the first pipe 61. This prevents the blood from coagulating in the guide tube 10 to maintain the smooth movement of the holding member 4. An anesthetic for local anesthesia, a physiological salt solution, or the like, for example, may be filled in the insufflator 69 so as to be infused into an organism through the guide tube 10. The aspirator 66 may aspirate the blood, the anesthetic, and the physiological salt solution, which remain in the guide tube 10.

Moreover, as in Modified Example 3 shown in FIG. 7, a partition wall 10a for partitioning the inside of the guide tube 10 may be provided inside the guide tube 10 to form a first room R1 and a second room R2. The first linear part 17 is inserted through the first room R1 while the second linear portion 19 is inserted through the second room R2. This prevents, in moving one of the linear portions, the other linear portion from obstructing the one linear portion to facilitate the operation.

In the case where the guide tube 10 is divided into the two rooms R1, R2, the second room R2 may be formed larger than the first room R1 as in Modified Example 4 shown in FIG. 8. In this case, the second linear portion 19, which is inserted through the relatively large second room R2, is moved easier than the first linear portion 17, which is effective in the case where the second linear portion 19 is moved mainly.

Furthermore, as in Modified Example 5 shown in FIG. 9, the distal end part of the guide tube 10 may be bent. The bending angle may be set arbitrarily. With this arrangement, only change in direction of the distal end of the guide tube 10 changes the direction that the first linear portion 17 and the second linear portion 19 extend.

In addition, as in Modified Example 6 shown in FIG. 10, marks 17a, 19a may be provided in the first linear portion 17 and the second linear portion 19, respectively. The marks 17a, 19a are made of an X-ray non-transmissive material and are arranged at substantially regular intervals. Accordingly, the operator can recognize the marks 17a, 19a under radiography so as to grasp the positions of the linear portions 17, 19, the shape and the size of the loop 3, and the size of the target object W with reference to the marks 17a, 19a. Each entirety of the first linear portion 17 and the second linear portion 19 may be covered with an X-ray non-transmisive material.

### INDUSTRIAL APPLICABILITY

As described above, the holder in accordance with the present invention can be used for, for example, taking out from the outside of a body a stent or the like falling off from a blood vessel.

1. A holder, comprising:
a long tubular guide member to be inserted at one end part thereof into an organism and to be protruded at the other end part thereof from the organism; and
a long holding member of which one end part is inserted in the guide member to be guided into the organism and which forms a loop for holding a target object within the organism,
wherein the holding member includes: a first linear portion extending from the one end part of the holding member and protruded from the other end part of the guide member; a return portion continuing from one end part of the first linear portion and returned toward the other end part of the guide member; and a second linear portion continuing from the return portion and extending toward and protruded from the other end part of the guide member, and
a first operation part and a second operation part which are to be held and operated by an operator are respectively provided at parts of the first linear portion and the second linear portion which are protruded from the other end part of the guide member.

2. The holder of claim 1,
wherein the other end part of the guide member branches into a first pipe through which the other end part of the first linear portion is inserted and a second pipe through which the other end part of the second linear portion is inserted.

3. The holder in claim 2,
wherein the first pipe is provided with first fixing means for switching the first linear portion to and from a state that the other end part thereof is fixed to the first pipe from and to a state that the fixed state is released.

4. The holder of claim 3,
wherein the second pipe is provided with second fixing means for switching the second linear part to and from a state that the other end part thereof is fixed to the second pipe from and to a state that the fixed state is released.

5. The holder of claim 1,
wherein the first operation part and the second operation part are provided with identification parts for identifying the operation parts.

6. The holder of claim 1,
wherein the guide member is divided into an insertion part to be inserted into the organism and a protruded part to be protruded outside the organism, and the insertion part is softer than the protruded part.

7. The holder of claim 1,
wherein an aspirator for aspirating a liquid in the guide member is connected to the guide member.

8. The holder of claim 1,
wherein an insufflator for infusing a chemical solution into the guide member is connected to the guide member.

9. The holder of claim 1,
wherein the guide member is bent at a part on the one end part side thereof.

10. The holder of claim 1,
wherein a mark made of an X-ray non-transmissive material is provided at the one end part of the holding member.

11. The holder of claim 1,
wherein the inside of the guide member is divided into a room through which the first linear portion of the holding member is inserted and a room through which the second linear portion thereof is inserted.
